(19) United States
(12) Patent Application Publication
(10) Pub. No.: US 2026/0151641 A1
(43) Pub. Date: Jun. 4, 2026

(54) PULSED ELECTROMAGNETIC FIELDS TO TREAT NEUROTROPHIC KERATITIS

(71) Applicant: EPITECH MEDICAL, INC., Irvine, CA (US)

(72) Inventor: Kamran Siminou, Newport Coast, CA (US)

(21) Appl. No.: 19/407,284

(22) Filed: Dec. 3, 2025

Related U.S. Application Data
(60) Provisional application No. 63/727,784, filed on Dec. 4, 2024.

(51) Int. Cl. A61N 2/00

(52) U.S. Cl. CPC A61N 2/006

(57) ABSTRACT

A system for applying electromagnetic stimulation to a periocular region of an eye to treat neurotrophic keratitis, includes: an electromagnetic field generator producing a time-varying magnetic field with a pulse duration of about 200 microseconds to about 400 microseconds, a rate of change of about 10,000 Tesla per second to about 20,000 Tesla per second, and a pulse rate of about 1 Hz to about 50 Hz; and a positioner configured to align the output of the electromagnetic field generator with the cornea, enabling treatment that enhances nerve regeneration and epithelial integrity.

This application claims the benefit of priority to U.S. Provisional Application Ser. No. 63/727,784, filed Dec. 4, 2024.

### FIELD

The present invention relates to systems, devices, and methods for treating ocular conditions, specifically neurotrophic keratitis (NK), through the use of pulsed electromagnetic fields.

### BACKGROUND

Neurotrophic keratitis (NK) is a degenerative corneal disease characterized by impaired corneal nerve function, leading to poor healing, corneal epithelial defects, and potential vision loss. In neurotrophic keratitis the cornea loses its normal sensory nerves, leading to reduced or absent corneal sensation. When the cornea can't feel properly, it also can't heal properly. As a result, even minor injuries may go unnoticed and can progress to more serious damage. Anything that damages the trigeminal nerve (the nerve that supplies feeling to the cornea) can lead to neurotrophic keratitis. Causes include viral infections (e.g., herpes simplex, varicella zoster), surgical trauma, systemic diseases (e.g., diabetes), and chemical injuries.

Because sensation is reduced, the cornea becomes dry, healing slows down, and persistent epithelial defects can form. In advanced cases, corneal ulcers, thinning, or even perforation may develop. Paradoxically, symptoms may be mild or absent because the cornea lacks sensation. However, patients may notice redness, blurry vision, dry eye-like symptoms, and in later stages, significant vision loss.

Current treatments focus on symptom management by attempting to protect the cornea and helping it heal. Such treatments include lubricating eye drops/ointments and therapeutic contact lenses. However, there has not been a sufficient attempt to repair the damage to the corneal nerves that are the root cause of the disease. There is clearly a need for a treatment for NK that has lasting effects and not merely treating symptoms.

### SUMMARY OF THE DISCLOSURE

Recent studies suggest that pulsed magnetic stimulation can modulate biological processes, such as nerve repair and epithelial healing, by influencing cellular mechanisms. However, this has never been applied or attempted as a treatment for NK. Building upon technologies designed for other conditions unrelated to NK, the invention provides a novel approach of applying a unique protocol of magnetic pulses to specifically address the pathophysiology and root causes of NK.

In one embodiment, a system and method for non-invasive treatment of neurotrophic keratitis using magnetic pulses to enhance corneal nerve regeneration and epithelial integrity is provided. The system generates electromagnetic fields characterized by:
Field strengths between 0.1 Tesla and 3 Tesla.
Pulse durations between 200 and 400 microseconds.
A rate of change of field strength between 10,000 and 20,000 Tesla per second.
A pulse rate of 1 to 50 Hz.

The system may include a wearable or table-mounted device, configured to position the electromagnetic field generator near the affected eye. The method is particularly effective in treating NK by promoting corneal nerve repair. The method is further effective by promoting corneal nerve repair while also restoring the protective barrier function of the corneal epithelium.

Other features and advantages will be apparent from the following description of the various embodiments of the disclosure, which illustrate, by way of example, the principles of the disclosed devices and methods.

### DETAILED DESCRIPTION

In one example, the system comprises:
a pulsed magnetic stimulator (“PMS”) having e.g., a time-varying electromagnetic field generator, which can produce magnetic pulses tailored to stimulate corneal nerve and periocular epithelial tissues. Said PMS can generate fields with strengths of up to 3 Tesla, and can operate within pulse durations of about 200 to about 400 microseconds and a rate of change of 10,000-20,000 Tesla per second; and
a positioner for said PMS in which the position is configured for precise placement of the magnetic field generator of said PMS relative to the cornea, maintaining a distance of, e.g., up to 10 cm.

In a wearable option, the system can include a head-mounted device designed for convenience and patient compliance, resembling a virtual reality headset or night vision goggles containing said PMS incorporated into it.

One example of a method of treating a subject suffering from NK is as follows:
Preparation—the subject's periocular region is positioned near the electromagnetic field generator of said PMS, ensuring the electromagnetic field generator aligns with the affected cornea so that the pulsed electromagnetic radiation reaches the cornea and the corneal nerve;
Magnetic Stimulation—pulses from the electromagnetic field generator are delivered at a rate of 1-50 Hz.
In one embodiment, treatment sessions last between about 10 minutes and about 30 minutes, or e.g. 10 minutes, 15 minutes, 20 minutes, 25 minutes or 30 minutes, with magnetic pulses applied in trains interspersed by silent intervals. In another embodiment, treatment sessions may be less than 10 minutes, or up to 10 minutes.
Repeat treatments are conducted weekly or as needed based on clinical response.

Clinical Monitoring can be performed by (i) evaluating corneal nerve regeneration through imaging or clinical markers (e.g., nerve density restoration), and (ii) assessing epithelial healing using fluorescein staining or corneal permeability tests.

The therapeutic benefits of the systems, methods and devices described herein are as follows:
Corneal nerve regeneration: enhancing corneal nerve density, and reducing the risk of persistent epithelial defects.
Epithelial integrity: restoring the barrier function, decreasing the risk of infections, and promoting healing.
Pain reduction: improving patient comfort by addressing underlying nerve dysfunction.

Without limitation, devices such as those described in U.S. Pat. Nos. 8,246,529, 11,247,065 and 11,141,601 and International Application WO 2014/181327, all of which are incorporated herein by reference in their entireties for all purposes, may be used in the treatment methods and systems described in the present invention. In one example, the EpiTech Corneal Magnetic Stimulation Device used in ClinicalTrials.gov Identifier No. NCT03012698, which is incorporated herein by reference in its entirety for all purposes, may be used.

While the invention is susceptible to various modifications and alternative forms, specific examples thereof have been shown by way of example in the written description provided herein. It should be understood, however, that the invention is not to be limited to the particular forms or methods disclosed, but to the contrary, the invention is to cover all modifications, equivalents, and alternatives falling within the spirit and scope of the appended claims.

We claim:

1. A system for applying electromagnetic stimulation to a periocular region of an eye to treat neurotrophic keratitis, the system comprising an electromagnetic field generator producing an output comprising an electromagnetic field having a field strength of up to 3 Tesla and a pulse rate of about 1 Hz to about 50 Hz.

2. The system of claim 1, further comprising a positioner configured to align the output of the electromagnetic field generator with a cornea, thereby promoting corneal nerve regeneration.

3. The system of claim 1, wherein the electromagnetic field generator generates an electromagnetic field with a pulse duration of about 200 microseconds to about 400 microseconds and a rate of change of about 10,000 Tesla per second to about 20,000 Tesla per second.

4. A method for treating neurotrophic keratitis by generating a pulsed electromagnetic field with a field strength of up to 3 Tesla and a pulse rate of about 1 Hz to about 50 Hz;
positioning the electromagnetic field near the cornea of an eye; and
delivering the electromagnetic field to the cornea.

5. The method of claim 4, wherein the magnetic field is applied in magnetic pulses applied in trains interspersed by silent intervals.

6. The method of claim 4, wherein the electromagnetic field has a pulse duration of about 200 microseconds to about 400 microseconds and a rate of change of about 10,000 Tesla per second to about 20,000 Tesla per second.

7. A method for treating neurotrophic keratitis, comprising:
using a pulsed magnetic stimulator (PMS) to deliver pulses of electromagnetic stimulation to a corneal nerve of an eye of a patient, wherein said PMS generates an electromagnetic field with a field strength of up to 3 Tesla, and a pulse rate of about 1 Hz to about 50 Hz;
positioning the PMS near the cornea; and
delivering the electromagnetic field to the cornea, thereby promoting corneal nerve regeneration.

8. The method of claim 7, wherein the magnetic field is applied in magnetic pulses applied in trains interspersed by silent intervals.

9. The method of claim 7, wherein said PMS generates an electromagnetic field with a pulse duration of about 200 microseconds to about 400 microseconds and a rate of change of about 10,000 Tesla per second to about 20,000 Tesla per second.
